(19) United States
(12) Patent Application Publication
(10) Pub. No.: US 2013/0210754 A1
(43) Pub. Date: Aug. 15, 2013

(54) METHOD FOR PRODUCING AN ENRICHED EXTRACT FROM VITIS VINIFERA L. LEAVES

(71) Applicants: Uwe Scheuring, Ochsenhausen-Hattenburg (DE); Martin Langer, Ulm (DE); Bernd Plohmann, Oberhoefen (DE); Bjoern Feistel, Andernach (DE); Bernd Walbroel, Andernach (DE)

(72) Inventors: Uwe Scheuring, Ochsenhausen-Hattenburg (DE); Martin Langer, Ulm (DE); Bernd Plohmann, Oberhoefen (DE); Bjoern Feistel, Andernach (DE); Bernd Walbroel, Andernach (DE)

(21) Appl. No.: 13/700,453

(22) PCT Filed: May 20, 2011
(86) PCT No.: PCT/EP11/58282

§ 371 Date: Feb. 5, 2013

(30) Foreign Application Priority Data
May 28, 2010 (EP) 10164316.1

(51) Int. Cl. A61K 31/7048; A61K 31/704; A61K 31/352

(52) U.S. Cl. CPC A61K 31/7048; A61K 31/352; A61K 31/704
USPC 514/27

(57) ABSTRACT

The present application relates to a process for preparing an extract of leaves of Vitis vinifera L. comprising the following steps:
a) preparing a drug from leaves of Vitis vinifera L.
b) extracting the drug with an eluant selected from among water and mixtures of water with alcohol, to obtain a raw extract,
c) separating the raw extract from the extracted drug,
d) at least partly eliminating the eluant, to obtain a thick extract,
e) redissolving the thick extract in water,
f) separating off any insoluble constituents,
g) selectively concentrating secondary plant ingredients by
   i. a 2-phase extraction or
   ii. membrane filtration;
the extract that is obtainable therefrom, pharmaceutical preparations containing this extract and the use thereof.

The present invention relates to extracts from leaves of Vitis vinifera L., the preparation and use thereof.

The term venous weakness in common parlance refers to a syndrome involving water retention in the lower extremities and taking the form of malaise such as heavy, easily tired legs. The so-called pooling of water in the legs indicates an oedematous accumulation of water in the tissues and presupposes that water can pass through from the blood vessels. It must therefore be assumed that the permeability of the vessels is increased excessively.

Venous weakness can be regarded as an early stage of Chronic Venous Insufficiency (CVI). CVI, also known as chronic venous congestion syndrome or Chronic Vein Insufficiency, is based on a microcirculatory disorder of the blood vessels resulting from an obstruction to venous outflow. The risk of suffering from chronic venous insufficiency increases with age, obesity, history of vein inflammations, deep vein thrombosis and severe leg trauma. CVI is twice as common in women as in men.

An important factor in the occurrence of chronic venous insufficiency is the presence of pathologically high blood pressure in the superficial veins. Normally this is 20-30 mmHg, but as a result of venous thrombosis, primary or secondary pathological vein valves or a weakened muscle pump it increases to 60-90 mmHg. This high pressure is the cause of the anatomical, physiological and histological changes in the blood vessels that are responsible for later complications. Under physiological conditions the capillary bed is protected from extreme changes in pressure, as precapillary arterioles are able to contract reflexively. However, patients with chronically raised venous pressure appear to have lost this reflex, i.e. in the event of changes to the position of the body, pressure changes are passed on directly to the capillary bed.

In permanently raised venous pressure, changes occur that affect the capillary walls. These include a broadening and lengthening of the capillary bed, an increased endothelial surface, increased storage of collagen IV in the basal membrane and pericapillary storage of fibrin. The combination of increased venous pressure and abnormal capillaries with increased permeability leads to a flushing of water, large proteins and red blood cells into the interstitial space. The accumulating fibrin also appears to be less easily dissolved. At the same time there is a reduced exchange of gases, i.e. cutaneous hypoxia. This leads, among other things, to leukocyte activation.

A similar process is familiar from the acute phase reaction of inflammations. In these, the permeability of the vascular walls is increased for a few minutes by vascular mediators such as histamine, prostaglandins (e.g. PGE-2), kinins and serotonin. Typical triggers of an acute phase reaction of this kind are cytokines such as interleukin-1 (IL-1β), interleukin-6 (IL-6), tumour necrosis factor alpha (TNF-α), tumour growth factor beta (TGF-β) or interferon gamma which are released inter alia by endothelial cells, fibroblasts, macrophages, granulocytes and lymphocytes. These immunocompetent cells release the cytokines for example after damage to or dying off of adjacent cells. The cytokines pass through the bloodstream into the liver where, together with cortisol, they activate the organ to produce around 30 different acute phase proteins (APP). The APPs promote the wound healing processes and counteract tissue damage. The effects of the acute phase reaction are both local and systemic. The local effects are supposed to limit possible infections and eliminate xenobiotics (such as for example synthetically produced dyes, pesticides and chlorinated solvents). Dead or damaged cells are eliminated by phagocytosis and repair processes are initiated. By contrast, the systemic effects are defence reactions that are intended to ensure the survival of the organism as a whole, for example against endotoxins.

According to the Guidelines of the German Society for Phlebology (AWMF Guidelines Register No. 037/009) the following agents are used inter alia as adjuvant pharmacotherapeutics in the treatment of Chronic Venous Insufficiency: acetylsalicylic acid, diuretics, fibrinolytics (defibrotide, stanozolol, sulodexide), fibrinolysis boosters, iloprost, pentoxyphyllin, prostaglandin E1, saponins (extracts of Centella, Hippocastanus, Ruscus), benzarone, calcium dobesilate, naftazone, tribenoside, Ginkgo biloba and numerous substances belonging to the large group of flavonoids [Haysteen BH, The biochemistry and medical significance of the flavonoids. Pharmacol Ther 2002;96(2-3):67-202]. Flavonoids have been shown to have a clearly positive effect on the development of oedema [Martinez M J, Bonfill X, Moreno R M, Vargas E, CapellàD. Phlebotonics for venous insufficiency. Cochrane Database of Systematic Reviews 2005, Issue 3. Art. No.: CD003229. DOI: 10.1002/14651858.CD003229.pub2.].

It has been found that the syndrome of venous weakness occurs significantly less in wine-growing regions. This would tend to indicate that the plant Vitis vinifera L. must contain compounds that have an antagonistic effect on the disease. Traditionally, vine leaves have both been eaten as a vegetable and also taken medicinally in the form of decoctions and infusions for menopausal disorders or for treating haemorrhoids. In the French Pharmacopoeia an extract of red vine leaves is recommended as a vein tonic. For some years preparations containing an aqueous extract of red vine leaves that are used successfully in chronic venous insufficiency (CVI) have been on the market under the name Antistax® [Kiesewetter H, Koscielny J, Kalus U, Vix J M, Peil H, Petrini O, et al. Efficacy of orally administered extract of red vine leaf AS 195 (folia vitis viniferae) in chronic venous insufficiency (stages I-II). A randomized, double-blind, placebo-controlled trial. Arzneimittel-Forschung 2000;50(2):109-117]. The extract contained therein is characterised by a content of about 5% flavonoids. About 0.1% anthocyanins are present as another group of phenolic compounds.

In accordance with the fact that the vine leaf extracts known hitherto are well tolerated, the aim of the present invention was to provide a novel extract which is also suitable for use for the whole range of inflammatory diseases. Local anti-inflammatory effects may possibly be helpful along the entire gastrointestinal tract in the treatment or prophylaxis of inflammatory diseases. Gingivitis, parodontitis, stomatitis, oesophagitis and gastritis describe the inflammatory diseases of the upper part of the digestive tract. Lymphocytic colitis, ulcerative colitis and Crohn's disease are the best known examples of inflammatory diseases of the intestinal region.

By haemorrhoids are meant, in common parlance, the varicose vein-like or knot-like swelling or dilation of an arteriovenous vascular cushion located between the rectum and the sphincter muscle of the anus. This vascular cushion is also referred to as the plexus haemorrhoidalis. It consists of a network of arteries and veins which together with the sphincter muscle allows the anus to be closed tightly. However, when the term haemorrhoids is used this is usually a reference to haemorrhoidal complaints. Typical symptoms are slight bleeding, a feeling of pressure, pruritis, skin rash and, in the advanced stage, problems in controlling the release of intestinal gases and stool. The causes may be among other things varicose vein-like dilation of blood vessels close to the anus. A similar disease entitled perianal thrombosis (=false haemorrhoids) is caused by the formation of a blood clot (thrombus) in the veins of the anal skin and is also referred to as anal thrombosis. Perianal thromboses are often caused by sitting for lengthy periods, sitting on cold surfaces, intense or prolonged straining on defecation (e.g. with hard stools) and during pregnancy and childbirth. They may also be caused by violent coughing, physical exertion, dehydration resulting from not drinking enough and even sneezing. Here again, blood vessels may be dilated beyond the usual amount. Both in haemorrhoidal complaints and in perianal thrombosis, besides surgical measures, it is usual to administer suppositories with pain-relieving or anti-inflammatory active substances (Guidelines on haemorrhoidal complaints “www.uni-duesseldorf.de/AWMF/II/081-007p.htm” and Guidelines on anal thrombosis “www.derma/de/585.0.html”).

### PRIOR ART

For other parts of the plant Vitis vinifera L., for example the grape seeds, a number of concentration methods have already been described. However, whereas methods such as microwave extraction (CN 1102589C) only provide for low concentration factors by increasing the yield, extractions with supercritical fluids (CN 1107110C) are only suitable for lipophilic structures. CN 1454896 uses an acidic primary extraction in conjunction with a liquid-liquid extraction for concentrating oligomeric proanthocyanidines. EP 348781 describes a concentration method for oligomeric phenolic compounds from grape seeds by membrane filtration, whereas WO 2005036988 and EP 1909750 claim a chromatographic method of selectively concentrating polyphenols and depleting monomers. GB 934554 describes a warm alcoholic-aqueous extraction (50-80% EtOH) of red vine leaf and subsequent acidification to obtain the red colour. In order to eliminate chlorophyll and waxes, a liquid-liquid extraction using benzene or carbon tetrachloride is proposed, or alternatively reprecipitation in pure water. The precipitation of the tannins can be speeded up by salting out. This is followed by a purification step using liquid-liquid extraction with butanol or pentanol. From the resulting alcoholic extract phase a procyanidine fraction is optionally precipitated with ether, petroleum or benzene and this is then gently dried (in vacuo or in a spray tower). This process is very laborious and cost-intensive and the resulting product differs very markedly from the reference extract. Therefore there is a need for an alternative manufacturing process by which the product can be manufactured at lower cost.

The problem of the present invention was to provide alternative active extracts from vine leaves.

The problem is solved by a method of preparing an extract from leaves of Vitis vinifera L. comprising the following steps:

a) preparing a drug from leaves of Vitis vinifera L.

b) extracting the drug with an eluant selected from among water and mixtures of water with alcohol, to obtain a raw extract,

c) separating the raw extract from the extracted drug,

d) at least partly eliminating the eluant, to obtain a thick extract,

e) redissolving the thick extract in water,

f) separating off any insoluble constituents,

g) selectively concentrating secondary plant ingredients by
i. a 2-phase extraction or
ii. membrane filtration.

According to the invention leaves of Vitis vinifera L. are used. These may be used in principle in fresh or dried form.

Preferably the leaves are comminuted before the extraction.

In a particular embodiment of the present invention the drug is prepared by collecting leaves of Vitis vinifera L. at a time when the flavonoid content is at its peak, drying and comminuting the leaves and optionally cutting the leaves into pieces.

Extraction is carried out with water or mixtures of water and alcohol in order to obtain a raw extract. Amounts of 5 to 30 kg of extraction agent per 1 kg of dried drug are particularly suitable.

Preferably, water or an ethanol-water mixture containing 5 to 95% (V/V) of ethanol are used as eluant.

It is particularly preferred to use an eluant which contains, besides water, ethanol in an amount of 25 to 50% (V/V).

Preferably, the extraction is carried out at a temperature in the range from 40 to 80°, while a temperature range of 15 to 25° C. below the boiling temperature of the eluant used has proved particularly suitable.

The pressure prevailing during the extraction is non-critical over a wide range. However, the extraction will normally be carried out at a pressure in the range from 900 to 1500 mbar, preferably from 950 to 1100 mbar and in particular at ambient pressure, i.e. at about 1013 mbar.

The crude extract is separated from the extracted drug. The separation is normally carried out by filtration or other methods known to the skilled man. For example, it is convenient to carry out filtration through a perforated sieve plate with 4 mm holes and, downstream thereof, polyamide filter bags with a mesh size of 250 μm. Other suitable methods for this purpose are known to the skilled man.

Then the eluant is at least partly eliminated from the raw extract separated off, thus obtaining a thick extract. By partial elimination of the eluant is meant, within the scope of the present invention, the elimination of 30 to 99% (V/V) relative to the volume of eluant put in, preferably 75 to 98% (V/V) and in particular substantially total elimination of the eluant, i.e. elimination of at least 95% (V/V). Usually, the eluant will be eliminated by heating and/or under reduced pressure. Suitable methods and equipment for this purpose are known to the skilled man.

The thick extract thus obtained is redissolved in water and insoluble constituents are separated off.

Usually, the redissolving is carried out in an amount of 1 part to 10 parts water to an amount of 1 part of the raw extract obtained. The insoluble constituents are separated off by methods known in the art, such as filtration and/or centrifugation.

A selective concentration of secondary plant ingredients is then carried out, either by a 2-phase extraction or by membrane filtration.

The 2-phase extraction may be carried out as a solid phase extraction, while an adsorber resin has proved suitable as the solid phase. Suitable adsorber resins selected for example from among the non-ionic hydrophobic divinylbenzene copolymers, aliphatic ester polymers and formophenol polymers are commercially available under the names Amberlite®, Levatite®, Miontech® and Diaion®. The adsorber resins that are suitable for the process according to the invention are preferably characterised by an average inner pore size in the range from 300-700 Angström, preferably 400-500 Angström.

The substances that bind to the adsorber are eluted and used further; the non-bound substances are discarded.

Usually, the extraction will be carried out in a temperature range of 10 to 30° C. The feed solution used is normally adjusted to a pH of between 3 and 6, preferably between 4.5 and 5.5, particularly preferably 5.

The two-phase extraction may also be a liquid-liquid extraction, while preferably the second phase is a water-immiscible liquid and is selected from among the alcohols, ketones, alkanes or esters. In one particular embodiment the second phase is a water-immiscible alcohol, preferably a C₄-C₅-alcohol, preferably n-butanol. The fraction that passes into the second phase is used further and the aqueous phase is discarded.

Usually, the extraction is carried out with a dry substance content of the starting solution of between 3 and 30%, preferably between 10 and 20%, particularly preferably 15%.

In principle, the selective concentration may also be a membrane filtration, while membranes with an exclusion size of 30 to 2000 kiloDalton 9 kDa) have proved satisfactory.

The permeate is used further, while the retentate is discarded.

In a particular embodiment of the present invention, after the selective concentration has been carried out, the extract is dried, so as to obtain a dry extract of Vitis vinifera L. The drying is carried out using standard methods known in the art, such as for example spray belt drying. However, it is also possible to use the concentrated extract further without drying it beforehand.

The method according to the invention as described above produces extracts of Vitis vinifera L. in which the ratio of native drug to extract (DEVnative) is 12:1 to 40:1. The total flavonoid content of the extracts obtained according to the invention and the extracts according to the invention is usually at least 15% by weight, preferably at least 20% by weight, and the content of anthocyanins is at least 0.1% by weight, preferably at least 0.2% by weight, most preferably at least 0.3% by weight. In this way, an enhanced inhibition of permeability-triggering cytokines is achieved, by comparison with the reference extract (according to the prior art).

Accordingly, the present invention further relates to the extract prepared by one of the methods described hereinbefore.

In another aspect the present invention relates to an extract preparation containing an extract according to the invention and at least one physiologically acceptable adjuvant. Suitable physiologically acceptable adjuvants are known to the skilled man.

In another aspect the present invention relates to the use of an extract according to the invention or an extract preparation according to the invention for the production of medicaments, pharmaceutical preparations or nutritional supplements. These are prepared by conventional methods known in the art.

In another aspect the present invention relates to the use of an extract according to the invention or an extract preparation according to the invention for the treatment or prophylaxis of inflammatory diseases of the blood vessels or of the gastrointestinal tract, in venous microcirculatory disorders, in ischemic diseases, in haemorrhoidal diseases or in perianal thrombosis. The inflammatory disease of the blood vessels treated according to the invention is more particularly phlebitis. The venous microcirculatory disorder treated according to the invention is more particularly chronic venous insufficiency (CVI). The ischaemic diseases treated according to the invention are more particularly heart diseases, particularly myocardial infarct.

In another aspect the present invention relates to the use of an extract according to the invention or an extract preparation according to the invention for the treatment or prophylaxis of acute or chronic inflammation of the gastrointestinal tract or haemorrhoidal complaints or perianal thrombosis.

The uses for treatment according to the invention may be carried out by intravasal, oral, rectal or transdermal administration of the novel extract or of the novel extract preparation. The novel extracts or novel extract preparations have a topical or systemic effect.

The invention is hereinafter illustrated in more detail by the following non-restrictive Examples.

### METHODS OF MEASUREMENT USED

### Chemical Parameters

The content of flavonoids is determined by an HPLC method which detects the flavonoids isoquercitrin, quercetin-3-O-β-D-glucuronide and camphor oil-3-O-β-D-glucoside. The stationary phase used is a Superspher RP8 separation column with 4 μm particles, with dimensions of 125×4 mm. The isocratic separation is carried out at a flow rate of 1 ml/min in a column oven at 25° C. The eluant is made up of 107 parts tetrahydrofuran, 55 parts acetonitrile and 810 parts phosphoric acid (0.003 mol/l). Detection is carried out at 360 nm.

The content of anthocyanins is determined by a separate HPLC method analogously to Ph. Eur 6.4 Monograph 2394 “Fresh Bilberry fruit dry extract, refined and standardised”.

### Pharmacological in Vitro Model for Cytokine Inhibition

Human monocytes are stimulated at 37° C. and 5% CO₂ for 24 h with LPS (10 ng/ml). The extracts are added 30 min before the addition of the LPS to check whether they are able to influence the LPS stimulation. Measured concentrations of the extracts of between 10-300 μg/ml were tested. After 24 hours the supernatant solutions were removed, centrifuged and adjusted to the concentrations of PGE-2, IL-6, IL-1, and TNF-alpha by means of ELISAs/EIAs (Biotrend/Immunotools) according to the manufacturer's instructions.

### Example 1

Aqueous Reference Extract (Prior Art)

2 kg of the drug Vitis vinifera leaf Ph. Eur. are extracted with 24 litres of osmosis water at 75° C. in the percolator. The eluate is filtered and evaporated down under reduced pressure to a thick extract containing about 50% dry matter. The spissum is mixed with 4% silicon dioxide and dried at 50° C. in the vacuum drying cupboard. The extract obtained is characterised by a DEVnative of 5:1, a flavonoid content of 5.1 and an anthocyanin content of 0.15%.

[%] Inhibition of the release of

| TNF-alpha | IL-1β | IL-6 | PGE-2 |
|---|---|---|---|
| 50.4 ± 5.9 | 46.9 ± 1.9 | 42.5 ± 5.3 | 11.6 ± 5.5 |
| at 50 μg/ml | at 300 μg/ml | at 50 μg/ml | at 300 μg/ml |

### Example 2

Adsorber Extract According to the Invention (Water-Based)

2 kg of the drug Vitis vinifera leaf Ph. Eur. are extracted with 44 litres of osmosis water at 75° C. in the percolator. The eluate is evaporated down under reduced pressure to a thick extract containing about 50% dry matter. The spissum is stored in the refrigerator for 16 hours, and diluted back to a dry content of 10% with osmosis water. After 24 hours the tannins precipitated are filtered off and then the thin extract is applied to adsorber material XAD7HP. The charged adsorber is washed with osmosis water and then the extract component is eluted with ethanol. The ethanol phase is evaporated down to a dry content of >50%. The spissum obtained is dried natively at 50° C. in the vacuum drying cupboard. The extract obtained is characterised by a DEVnative of 19:1, a flavonoid content of 21.1 and an anthocyanin content of 0.77%.

[%] inhibition of the release of

| cytokine | IL-6 | PGE-2 |
|---|---|---|
| extract according to | 42.5 ± 4.6 | 32.1 ± 7.1 |
| Ex. 2 | at 10 μg/ml | at 50 μg/ml |
| extract according to | 42.5 ± 5.3 | 11.6 ± 5.5 |
| Ex. 1 | at 50 μg/ml | at 300 μg/ml |

For the adsorber extract according to Example 2 there was shown to be an increase in cytokine inhibition relative to the prior art according to Example 1.

In the case of IL-6 a concentration of the extract according to the invention that is lower by a factor of 5 is needed to achieve the same anti-inflammatory effect.

The effect on PGE-2 is even more marked. At a measured concentration of 300 μg/ml according to Example 1 only an 11.6% inhibition could be measured. The novel extract according to Example 2 achieved a 32.1% inhibition even with a 6 times lower concentration of 50 μg/ml.

These two are higher by a factor 5 or factor 18, respectively (3 times better % value×concentration factor 6) than the concentration factor of 3.8 of the novel extract as compared with Example 1. The concentration factor of 3.8 is obtained from the quotient of the DEVnative of the extracts being compared.

### Example 3

Extract According to the Invention (by Liquid-Liquid Extraction)

2 kg of the drug Vitis vinifera leaf Ph. Eur. are extracted with 44 litres of osmosis water at 75° C. in the percolator. The eluate is evaporated under reduced pressure to form a thick extract containing about 50% dry matter. The spissum is refrigerated for 16 hours and rediluted to a dry content of 10% with osmosis water. After 24 hours the tannin fraction precipitated is filtered off and then the thin extract is twice treated with n-butanol in a ratio of 3:2. The butanol phase is evaporated down to a dry content of >50%. The spissum obtained is dried natively in the vacuum drying cupboard at 50° C. The extract obtained is characterised by a DEVnative of 16:1, a flavonoid content of 21.7% and an anthocyanin content of 0.43%. The aqueous phase remaining is characterised by a DEVnative of 5:1, a flavonoid content of <0.1% and an anthocyanin content below the detection limits.

| | [%] Inhibition of the release of | | |
|---|---|---|---|
| | TNF-alpha | IL-1β | PGE 2 |
| BuOH phase | 52.2 ± 6.7 | 55.8 ± 6.6 | 24.3 ± 3.9 |
| Ex. 3A | at 10 μg/ml | at 10 μg/ml | at 50 μg/ml |
| H₂O phase | 17.1 ± 4.8 | 39.8 ± 6.0 | 1.6 ± 4.6 |
| Ex. 3B | at 50 μg/ml | at 300 μg/ml | at 300 μg/ml |

A comparison of the two phases from the liquid-liquid treatment clearly shows that the anti-inflammatory principles can be concentrated in the organic phase (butanol). For the extract concentrated with butanol according to Example 3A there was found to be an increase in cytokine inhibition of factor 15 (TNF-a), factor 42 (1L-1β) and factor 91 (PGE-2) compared with the aqueous phase separated off according to Example 3B.

This compares with a concentration factor of only 3.2 (quotient of the DEV native of the extracts in Examples 3A and 3B that are to be compared).

This underlines the selective accumulation of the active principles from the aqueous phase into the organic phase.

The novel extract according to Example 3A is also superior to the prior art.

[%] Inhibition of the release of

| cytokine | TNF-alpha | PGE 2 |
|---|---|---|
| Extract according to | 52.2 ± 6.7 | 24.3 ± 3.9 |
| Ex. 3A | at 10 μg/ml | at 50 μg/ml |
| Extract according to | 50.4 ± 5.9 | 11.6 ± 5.5 |
| Ex. 1 | at 50 μg/ml | at 300 μg/ml |

The extract according to Example 3A was shown to increase the cytokine inhibition compared with the prior art according to Example 1.

With TNF-alpha it is necessary to have a concentration of the novel extract 3A that is 5 times lower in order to obtain the same anti-inflammatory effect.

The effect on PGE-2 is even more marked. At a measured concentration of 300 μg/ml according to Example 1, only an 11.6% inhibition could be measured. The novel extract according to Example 2 achieved a 24.3% inhibition even with a 6 times lower concentration of 50 μg/ml.

These two are higher by a factor 5 or factor 12, respectively (2 times better % value×concentration factor 6) than the concentration factor of 3.2 of the novel extract as compared with Example 1. The concentration factor of 3.2 is obtained from the quotient of the DEV native of the extracts being compared.

### Example 4

Preparation of an Ethanolic Extract According to the Invention

2 kg of the drug Vitis vinifera leaf Ph. Eur. are extracted with 24 litres of EtOH 40% V/V at 45° C. in the percolator. The eluate is filtered and evaporated down under reduced pressure to form a thick extract containing about 50% dry matter. The spissum is refrigerated for 16 hours, and rediluted with osmosis water to a dry content of 10%. After 24 hours the tannins precipitated are filtered off and then the thin extract is freed from other coextracted high molecular substances by membrane filtration (MWCO 300 kDa). The resulting fraction (permeate) is brought to dryness at 50° C. in vacuo. The extract obtained is characterised by a DEVnative of 6.7:1 and a flavonoid content of 7.6%.

### Example 5

Preparation of an Adsorber Extract According to the Invention (EtOH-Based)

2 kg of the drug Vitis vinifera leaf Ph. Eur. are extracted with 24 litres of EtOH 20% V/V at 75° C. in the percolator. The eluate is evaporated down under reduced pressure to form a thick extract containing about 50% dry matter. The spissum is refrigerated for 16 hours, and rediluted with osmosis water to a dry content of 10%. After 24 hours the tannins precipitated are filtered off and then the thin extract is applied to adsorber material XAD7HP. The charged adsorber is washed with osmosis water and then the extract component is eluted with ethanol. The ethanol phase is evaporated down to a dry content of >50%. The spissum obtained is dried natively at 50° C. in the vacuum drying cupboard. The extract obtained is characterised by a DEVnative of 21:1 and a flavonoid content of 25.1%.

1. (canceled)

2. The process according to claim 16, wherein the eluant is water or an ethanol-water mixture that contains 5-95% (V/V) ethanol.

3. The process according to claim 16, wherein the eluant is heated to a temperature of 40-80° C., prior to extracting.

4. The process according to claim 16, wherein the 2-phase extraction is a solid phase extraction.

5. The process according to claim 4, wherein the second phase is an adsorber resin selected from among the non-ionic hydrophobic divinylbenzene copolymers, aliphatic ester polymers and formophenol polymers.

6. The process according to claim 16, wherein the two-phase extraction is a liquid-liquid extraction.

7. The process according to claim 6, wherein the second phase is a water-immiscible liquid selected from alcohols, ketones, alkanes and esters.

8. An extract prepared according to claim 16.

9. The extract according to claim 8, wherein the total flavonoid content is at least 15% by weight and the content of anthocyanins is at least 0.1% by weight.

10. The extract according to claim 8 together with at least one physiologically acceptable adjuvant.

11. (canceled)

12. (canceled)

13. (canceled)

14. (canceled)

15. (canceled)

16. A process for preparing an extract of leaves of Vitis vinifera L. comprising the following steps:
a) collecting leaves of Vitis vinifera L.;
b) extracting material from such collected leaves with an eluant selected from water and mixtures of water and alcohol to obtain a raw extract;
c) at least partially eliminating the elutant from the raw extract to obtain a thick extract;
d) dissolving the thick extract in water;
e) removing any insoluble constituents from the re-dissolved thick extract; and
f) selectively concentrating the resulting re-dissolved thick extract to concentrate its components by either two-phase extraction or membrane filtration.
